(19) United States
(12) Patent Application Publication
(10) Pub. No.: US 2024/0336960 A1
(43) Pub. Date: Oct. 10, 2024

(54) METHODS FOR MULTI-DIMENSIONAL LABELING OF NUCLEIC ACIDS OF A CELLULAR SAMPLE IN SITU

(71) Applicant: Scale Biosciences Inc., San Diego, CA (US)

(72) Inventors: Frank STEEMERS, Encinitas, CA (US); Robert R. STICKELS, San Carlos, CA (US)

(21) Appl. No.: 18/576,084

(22) PCT Filed: Jul. 19, 2022
(86) PCT No.: PCT/US2022/037583

§ 371 Date: Jan. 2, 2024

Related U.S. Application Data
(60) Provisional application No. 63/224,135, filed on Jul. 21, 2021.

(51) Int. Cl. C12Q 1/6841; G01N 33/58

(52) U.S. Cl. CPC C12Q 1/6841; G01N 33/58; G01N 2458/10

(57) ABSTRACT

Provided herein, among other things, is a method to spatially-label nucleic acid barcodes in or on a cellular sample in situ. In some embodiments, the method may comprise: obtaining a cellular sample comprising analytes or derivatives, fixing the analytes to their native location, providing a population of barcoded nucleic acids, and applying a stimulus to distribute the barcoded nucleic acids over the sample thereby differentially labeling areas and analytes of the sample. This process may include repeating the steps one or more times in various dimensions.

### CROSS-REFERENCING

This application claims the benefit of U.S. provisional application Ser. No. 63/224,135, filed on Jul. 21, 2021, which application is incorporated by reference herein for all purposes.

### BACKGROUND

Single cell analysis has become a standard tool for studying how genes are regulated, and how cellular states and cellular functions are defined. However, gene expression in individual cells is influenced by their location within a tissue. As such, in order to gain a more complete understanding of a cell, one should obtain information about gene expression in individual cells in their morphological context. Spatial single cell analysis is enabling scientists to obtain a holistic understanding of cells in their morphological and tissue context, i.e., cell state is determined by neighboring cells (Srivatsan et al. Embryo-scale, single-cell spatial transcriptomics, Science 2021). Methods have been proposed to label cells with unique barcodes that both encode the spatial location and the analytes of each individual cell in the tissue. One approach is to provide spatially organized arrayed barcodes to the cellular sample, and transferring the analytes to the array thereby labeling the spatial location of the cells and the analytes of interest. As such, spatial single cell information can be obtained, and in some methods across a limited depth of the tissue. However, all current methods ignore the multi-layer dimension of cellular samples, have significant inefficiencies in respect to capture of the cells and analytes of interest, and have limited single cell resolution. Tissues and cellular samples consist of multi-cellular layers and function and the identity of each cell is defined by their respective neighboring cells.

This disclosure provides a way to add spatially-addressable barcodes to cells and their analytes in situ (i.e., within the tissue section) across multiple cellular layers. After barcoding the cellular analytes, the derived libraries are sequenced, the sequences can be mapped to a site in the tissue section using the sequenced barcode. This method can be used to provide profiles for single cells across many cellular layers that are in a tissue section and thus solves the problems discussed above. The methods provided herein can therefore be useful for research, diagnostic purposes, e.g., for the diagnosis of cancer, and possibly aid in the selection of targeted therapies.

### SUMMARY

Provided herein, among other things, are methods for multi-dimensional labeling of nucleic acids of a cellular sample in situ. Analogous to gel electrophoresis, barcoded nucleic acids with different mobilities are distributed over a sample thereby uniquely labeling areas of interest. Distribution can occur through an external stimulus, for example a current through the cellular sample. In some embodiments the method may comprise: obtaining a cellular sample comprising analyte molecules that are fixed to their native position, barcoding the analyte molecules with barcoded nucleic acids through a process of transferring and distributing barcoded nucleic acids over the sample in a specific direction (e.g. z-direction to differentially label multi-layers of sample) by selectively applying an external stimulus (e.g., an electrochemical stimulus), resulting in the spatial distribution of barcoded nucleic acids to the sample, removing any unreacted barcoded nucleic acids from the sample, and optionally repeating the barcoding more times and in two or more dimensions. The addition step of the barcoded nucleic acid to the analyte of interest can be done enzymatically (using a polymerase, ligase, terminal transferase, etc.) or chemically, in a templated or non-templated manner.

In some embodiments, the method may comprise: (a) obtaining a cellular sample comprising nucleic acid analyte molecules; (b) contacting the sample with a population of barcoded nucleic acids; (c) applying a stimulus to distribute the barcoded nucleic acids through the sample, resulting in differentially labeling of the cellular sample, (d) reacting the barcoded nucleic acids with the nucleic acid analyte molecules, in situ, to produce barcoded nucleic acid products; (e) extracting the barcoded nucleic acid products from the sample; and (f) analyzing the extracted barcoded nucleic acid products.

The method can, for example, be used to add a spatially addressable barcodes to cDNA molecules that are made in situ. In this embodiment, the barcoded cDNA may be sequenced, and the cDNA sequences can be mapped to a physical position on the sample by the barcodes associated with those sequences.

### BRIEF DESCRIPTION OF THE FIGURES

The skilled artisan will understand that the drawings described below are for illustration purposes only. The drawings are not intended to limit the scope of the present teachings in any way.

FIG. 1 illustrates some of the principles of an embodiment of the present method.

FIG. 2 spatial barcoding along any axis with a variety of encoders, e.g., labeling in the x, y, or z direction of a cellular sample.

FIG. 3 illustrates an example of how barcodes can be distributed over a sample. Arrayed and barcoded oligonucleotides (oligo-BC1 etc.) can be transferred to the cellular sample, thereby differentially labeling regions of interest.

FIG. 4 Example workflow for spatially labeling cellular samples.

FIG. 5 illustrates how a barcoded nucleic acid can be attached to cDNA, transposed DNA, or antibody oligonucleotide tag in situ.

FIG. 6 illustrates some of the design principles for the method illustrated in Example 6.

### DEFINITIONS

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of ordinary skill in the art with the general meaning of many of the terms used herein. Still, certain terms are defined below for the sake of clarity and ease of reference.

As used herein, the term “spatially addressed” and “spatially addressable” refer to sequences that can be mapped to a site or position on a sample, e.g., by x-y-z coordinates.

As used herein, the term “nucleic acid barcode or barcode” refers to a sequence of nucleotides that is appended onto one or more target molecules either directly via covalent coupling to the target molecule, or indirectly via hybridization, polymerization, or ligation to a pre-installed nucleic acid anchor sequence. A nucleic acid barcode can be at least four nucleotides, e.g., 4-20 nucleotides, in length. The barcode can contain a common sequence, a primer, or UMI or combination thereof.

As used herein, the term “spatially addressed nucleic acid barcode” refers to sequences of nucleotides that are appended onto one or more target molecules, where the sequence appended onto each target molecule indicates a position on the sample, e.g., by x-y-z coordinates. A sample that contains spatially addressed nucleic acid barcodes can be subdivided into multiple areas, where each area is associated with a different barcode sequence.

As used herein, the term “analyte” or “target molecule” refers to any molecule within the cell for which identity, abundance, and spatial information is being queried. Examples of target molecules or analytes include nucleic acids (genomic DNA, chromosomal DNA, RNA, mRNA, and other intracellular nucleic acids; proteins and polypeptides; carbohydrates; and metabolites. As used herein, the term “nucleic acid analyte molecules” refers to any nucleic acid molecule representing the original analyte molecule as a DNA copied or encoded representation. For instance, in the case of mRNA, a derivative nucleic acid analyte molecule would be a cDNA molecule; in the case of a protein molecule, one example of a derivative nucleic acid analyte molecule is the DNA tag on a bound antibody to its cognate protein.

As used herein, the term “anchor sequence” refers to a nucleic acid sequence attached to a target molecule either directly via covalent conjugation, or indirectly via binding of an affinity agent (e.g., antibody, nanobody, binding agent, aptamer, etc.) with an attached nucleic acid sequence minimally capable of accepting a barcode sequence. The barcode sequence may include a constant region complementary to the anchor adapter sequence, and contains a barcode portion representing a spatial barcode, or a single cell barcode, or a sample index barcode, or a UMI sequence, or any other sequence of utility in library construction, or any combination thereof. The anchor sequence may be comprised of a constant sequence for priming or amplification purposes, a UMI sequence, a general barcode sequence (e.g., sample index, analyte index, etc.) and an adapter sequence mediating addition of the barcode sequence via hybridization and templated primer extension or ligation, in some cases a bridging or splint oligo may be used to attach the barcode sequence to the adapter.

As used herein, the term “cellular sample” is intended to include samples are made by, e.g., growing cells on a planar surface, samples that are made by depositing cells on a planar surface, e.g., by centrifugation, samples that are made by cutting a three-dimensional object that contains cells into sections and mounting the sections onto a planar surface, i.e., producing a tissue section, as well as tissue blocks that are not cut prior to processing. The surface upon which a sample may be mounted may be, e.g., glass, metal, ceramic, plastic, etc.). If the sample is fixed, it may be fixed using any number of reagents including formalin, methanol, paraformaldehyde, methanol: acetic acid, glutaraldehyde, bifunctional crosslinkers such as bis (succinimidyl) suberate, bis (succinimidyl) polyethyleneglycol, and any other fixative which irreversibly or reversibly bonds with biomolecules in the cellular sample. A section (e.g., a cryosection) of a tissue sample (e.g., of a fresh frozen tissue sample) that has a thickness in the range of 1-50 μm (e.g., in the range of 1-5 μm or 5-20 μm) is an example of a cellular sample, although there are many alternatives. In some embodiments the cells in the sample may be fixed and/or permeabilized, e.g., using a detergent or a solvent. The term cellular samples also include whole tissues, embedded tissue, embedded and cleared tissue, or parts thereof.

As used herein, the term “tissue section” refers to a piece of tissue that has been obtained from a subject and mounted on a planar surface, e.g., a microscope slide.

As used herein, the term “formalin-fixed paraffin embedded (FFPE) tissue section” refers to a piece of tissue, e.g., a biopsy sample that has been obtained from a subject, fixed in formaldehyde (e.g., 3%-5% formaldehyde in phosphate buffered saline) or Bouin solution, embedded in wax, cut into thin sections, and then mounted on a microscope slide.

As used herein, the term “nucleic acid modification” is a modification to a nucleic acid molecule that alters the migration of the nucleic acid molecule over the cellular sample. A modification may be on the 3′ end, 5′ end or internal modification of the nucleic acid molecule.

As used herein, a stimulus or external stimulus, may be light or an electrochemical, pH change, a gradient (e.g., sucrose gradient) or current. The stimulus is used to distribute the barcodes over the cellular sample.

The term “nucleic acid modification” is intended to refer to the types of groups that are used to modify the diffusion constant, electrophoretic migration in, for example, gel electrophoresis methods (at least some of which are described in Lee et al. Agarose Gel Electrophoresis for the Separation of DNA Fragments J Vis Exp. 2012; (62): 3923). Examples that can affect mobility include, but not limited, length of the nucleic acid, charged groups, bulky groups, secondary structure of nucleic acids, ability to complex with another molecule, affinity tags etc. An example of an affinity tag altering electrophoresis migration is described in Abrams et al. patent WO1999045374A2, Purification and detection processes using reversible affinity electrophoresis.

Other examples were modification changes the mobility in electrophoresis are described in Carruthers et al. Protein Mobility Shifts Contribute to Gel Electrophoresis Liquid Chromatography Analysis J Biomol Tech. 2015 September; 26 (3): 103-112., for example, mobility shifts during SDS-PAGE fractionation occur with chemical modification of proteins, including phosphorylation, glycosylation, hydroxylation, methylation, and ubiquitination.

As used herein, barcode or barcodes refers to nucleic acids (four or more nucleotides), a population of nucleic acids, RNA, DNA, protein, antibody or derivative, or any kind of label that can be distributed over the cellular sample using a ‘stimulus’ and thereby labeling the analytes or derivatives of interest.

As used herein, the term “electrophoresis” is the movement of charged particles in a fluid or gel under the influence of an electric field. A review on electrophoresis and related materials is REVIEW ON ELECTROPHORESIS, CAPILLARY ELECTROPHORESIS AND HYPHENATIONS S. Gummadi et al. IJPSR (2020), Volume 11, Issue 12. In some examples, pulsed field electrophoresis can be used. Pulsed field gel electrophoresis is a technique used for the separation of DNA molecules by applying to a gel matrix an electric field that periodically changes direction. In some examples, the analyte is labeled during or after electrophoresis, i.e. during pulsed-field electrophoresis to maintain the spatial location of the barcode, minimizing diffusion.

As used herein, the term “deprotecting” refers to the removal of the blocking group from a reversible terminator. Deprotection allows the nucleic acid on which the reversible terminator is present to be extended. If the reversible terminator is on the 3′end, deprotecting will typically result in a 3′ hydroxyl, thereby allowing the unblocked nucleic acid to be extended by an enzyme such as a polymerase or terminal transferase. If the reversible terminator is on the 5′ end, deprotecting (or “de-blocking” as it may be called in oligonucleotide synthesis methods) will typically result in a 5′ terminal hydroxyl, that can be reacted with nucleoside phosphoramidite or nucleoside H-phosphonate, thereby allowing the unblocked nucleic acid to be extended via chemical addition.

As used herein, the term “reversible terminator nucleotide” refers to 3′-O-blocked, 3′-unblocked and other reversible terminator deoxynucleotides that are reversibly blocked at the 3′ end (see, e.g., Chen at al Genomics, Proteomics & Bioinformatics 2013 11:34-40). These reversible terminators typically have a 5′ triphosphate and a cleavable blocking group that prevents addition onto the 3′ end. The term “reversible terminator deoxynucleotide” also refers to nucleoside phosphoramidite and nucleoside H-phosphonates used in synthetic oligonucleotide synthesis methods.

As used herein, the term “removing” refers to any action that results of the elimination of a compound. Removing may include degrading, inactivating, or washing away, or any combination thereof.

As used herein, the term “5′ tail”, in the context of a tailed oligonucleotide, refers to a 5′ part of an oligonucleotide that is not complementary to a target and does not hybridize to the target that the 3′ hybridizes to. A tail can be as long as needed, e.g., in the range of 20-100 bases, as desired.

As used herein, the term “oligonucleotide” refers to a multimer of at least 2 nucleotides, e.g., at least 5, at least 10, at least 15 or at least 30 nucleotides. In some embodiments, an oligonucleotide may be in the range of 15-200 nucleotides in length, or more. Any oligonucleotide used herein may be composed of G. A. T and C, or bases that are capable of base pairing reliably with a complementary nucleotide. In some embodiments, an oligonucleotide may additionally contain one or more “universal” bases that can base pair with any of G. A. T and C. Universal bases include 2′-deoxyinosine 2′-deoxynebularine, 3-nitropyrrole 2′-deoxynucleoside and 5-nitroindole 2′-deoxynucleoside, although others are known. Oligonucleotides may be synthetic or may be made enzymatically, and, in some embodiments, are 30 to 150 nucleotides in length. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example.

The term “hybridization” or “hybridizes” refers to a process in which a nucleic acid strand anneals to and forms a stable duplex, either a homoduplex or a heteroduplex, under normal hybridization conditions with a second complementary nucleic acid strand and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing. A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions includes hybridization at about 42C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42°° C.

The term “sequencing”, as used herein, refers to a method by which the identity of at least 2 consecutive nucleotides (e.g., the identity of at least 5, at least 10, at least 50 or at least 100 or more consecutive nucleotides) of a polynucleotide are obtained.

The term “next-generation sequencing” refers to the so-called parallelized sequencing-by-synthesis or sequencing platforms currently employed by, e.g., Illumina, Life Technologies, BGI Genomics (Complete Genomics technology), Ultima Genomics, Singular Genomics, Element genomics, PacBio, Oxford Nanopore, and Roche etc.

The term “duplex,” or “duplexed,” as used herein, describes two complementary polynucleotides that are base-paired, i.e., hybridized together.

The terms “determining,” “measuring,” “evaluating,” “assessing,” “assaying,” and “analyzing” are used interchangeably herein to refer to forms of measurement, and include determining if an element is present or not. These terms include both quantitative and/or qualitative determinations. Assessing may be relative or absolute.

The term “ligating”, as used herein, refers to the enzymatically catalyzed joining of the terminal nucleotide at the 5′ end of a first DNA molecule to the terminal nucleotide at the 3′ end of a second DNA molecule.

The terms “plurality”, “set” and “population” are used interchangeably to refer to something that contains at least 2 members. In certain cases, a plurality may have at least 10, at least 100, at least 100, at least 10,000, or at least 100,000 members.

A “primer binding site” refers to a site to which an oligonucleotide hybridizes in a target polynucleotide or fragment. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds.

The term “extending”, as used herein, refers to the extension of a nucleic acid by the addition of nucleotides using a polymerase or by a chemical reaction.

As used herein, the term “splint” refers to an oligonucleotide that hybridize to the ends of two other polynucleotides.

The term ‘UMI’ as used herein refers to a unique molecular identifier to make a library unique or substantially unique. UMIs are typically added onto nucleic acid molecules by introducing a random sequence (e.g., a random 6-mer or the like) to the molecules.

Other definitions of terms may appear throughout the specification.

### DETAILED DESCRIPTION

Provided herein is a method for spatially labeling analytes in or on a cellular sample in situ. In some embodiments, the method may comprise: (a) obtaining a cellular sample (e.g., a tissue or tissue section) comprising nucleic acid analyte molecules; (b) contacting the sample with a population of barcoded nucleic acids; (c) applying a stimulus to distribute the barcoded nucleic acids through the sample (e.g., in the x, y or z plane), resulting in differentially labeling of the cellular sample; (d) reacting the barcoded nucleic acids with the nucleic acid analyte molecules, in situ, to produce barcoded nucleic acid products (e.g., by ligation, extension, hybridization, chemical ligation, etc.); (e) extracting the barcoded nucleic acid products from the sample; and (f) analyzing the extracted barcoded nucleic acid products. The method may further comprise sequencing the barcodes produced in step (f) and at least part of the nucleic acid molecules to which they are attached, or an amplification product thereof. The method may further comprise mapping the sequenced nucleic acid molecules to a site in or on the cellular sample using the barcode to which it is attached.

The barcoded nucleic acids may be distributed through the sample via any suitable method, e.g., through diffusion or active transportation (e.g., electrophoresis, magnetic attraction, etc.), and the external stimulus may be is a voltage differential (electrophoresis), magnetism, temperature, light, pH, etc. or any combination thereof. Further, the barcoded nucleic acids may be distributed through the sample in the x, y or z plane, or any combination thereof.

In some embodiments, the nucleic acid molecules may be endogenous (e.g., may be chromatin, genomic DNA, or RNA, etc.), produced in situ (e.g., cDNA or fragments of genomic DNA) or added to the sample (e.g., by binding to the sample to antibody-oligonucleotide conjugates). In some embodiments, the cellular sample may be made by hybridizing, ligating or binding a barcoded oligonucleotide to a sample that contains cells or cellular material (e.g., oligonucleotides or probes hybridized to RNA or DNA), including cleared versions of the same. In these embodiments, the cellular sample may be modified prior to barcoding by transposition, cDNA synthesis, oligo affinity tagging of proteins, etc.

In any embodiment, the barcoded nucleic acids may have different diffusion constants or electrophoretic mobility, which allows the barcoded nucleic acids to migrate through the sample in accordance with their respective diffusion constants or electrophoretic mobility. For example, in some embodiments, the population of barcoded nucleic acids comprises barcoded nucleic acids of different lengths and the barcodes indicate the length of the nucleic acid in which it is present. In these embodiments, in step (c) the barcoded nucleic acids the shorter barcoded nucleic acids migrate through the sample further than longer barcoded nucleic acids. In this example, the wherein barcoded nucleic acids become distributed through the sample according to their lengths.

In any embodiment, the cellular sample may be made by: (i) embedding the cellular sample in a matrix, (ii) fixing the analytes to the matrix, (iii) optionally clearing of cellular materials, where the matrix provides a medium for transporting barcoded nucleic acids an/or attachment of analytes. In any embodiment, the barcoded nucleic acids may bind to an endogenous molecule or nucleic acid that is hybridized to an endogenous molecule.

In some embodiments, the barcoded nucleic acids may present on a surface that becomes in contact with the sample. The barcoded nucleic acids them migrate from the surface into the sample, becoming separated from one another on the way.

The barcoded nucleic acids with the nucleic acid analyte molecules may react by any suitable method, e.g., by ligation, extension, hybridization, chemical ligation, etc. This step may be done enzymatically or chemically (i.e., in an enzyme-free way). The addition of the barcoded nucleic acids is templated or non-templated, as desired. In some embodiments, the barcoded nucleic acid may have a sequence at the 3′ end that hybridizes to a nucleic acid analyte and a 5′ barcode sequence, and the 3′ end of the barcoded nucleic acid is extended using the analyte as a template, thereby copying the complement of the analyte into the barcoded nucleic acid by primer extension. In some embodiments, step (d) may comprise a ligation, primer extension, gap-fill/ligation, hybridization, and/or chemical ligation. In some embodiments, the barcoded nucleic acid molecules may be immobilized in the sample prior to the reaction (e.g., by hybridization to another molecule).

In any embodiment, the number of barcoded nucleic acids in the population may comprise at least 2, at least 5, at least 10, at least 100, at least 1000, at least 10,000, at least 100,000, at least 1M nucleic acids, where the different barcoded nucleic acids migrate into the sample at different rates (e.g., in accordance with their length). For example, in some embodiments, the population may contain at least two barcoded nucleic acids, which labels two different regions in the sample.

In some embodiments, the external stimulus is selectively applied to the sample in one direction (e.g., a voltage differential, which should, for example, separate molecules that have different lengths in situ). After a period of time, a different external stimulus may be applied to a second direction (e.g., magnetism, which should, for example, separate molecule that have different magnetisms). The barcoded nucleic acids may differ in nature based on oligonucleotide length, modification, chemical group or combination thereof. In some embodiments, the labeling method may be repeated one or more times using external stimuli applied from different directions.

In some embodiments, the cellular sample may be made by i, hybridizing a tailed reverse transcription primer to RNA in the cellular sample; ii, binding a barcoded oligonucleotide to the reverse transcription primer in situ to produce barcoded first strand cDNA. In these embodiments, the barcoded oligonucleotides may be coupled to the cDNA (e.g., by ligation) or the barcoded oligonucleotide may be extended using the cDNA as a template, thereby producing barcoded cDNA molecules. In some embodiments, the barcoded nucleic acid contains a common sequence at the 3′ end, e.g., oligodT. In other embodiments, the barcoded nucleic acid may have a 3′ end that is capable of random priming, or gene-specific sequence.

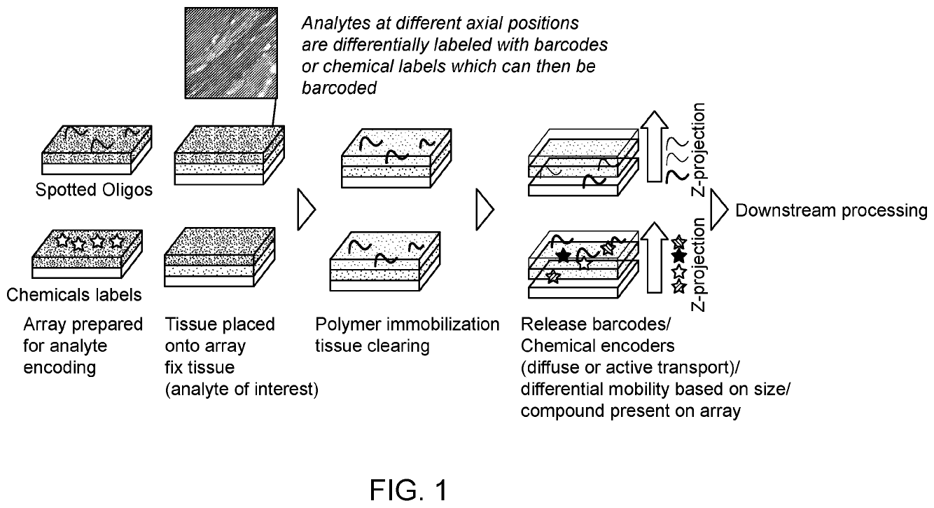

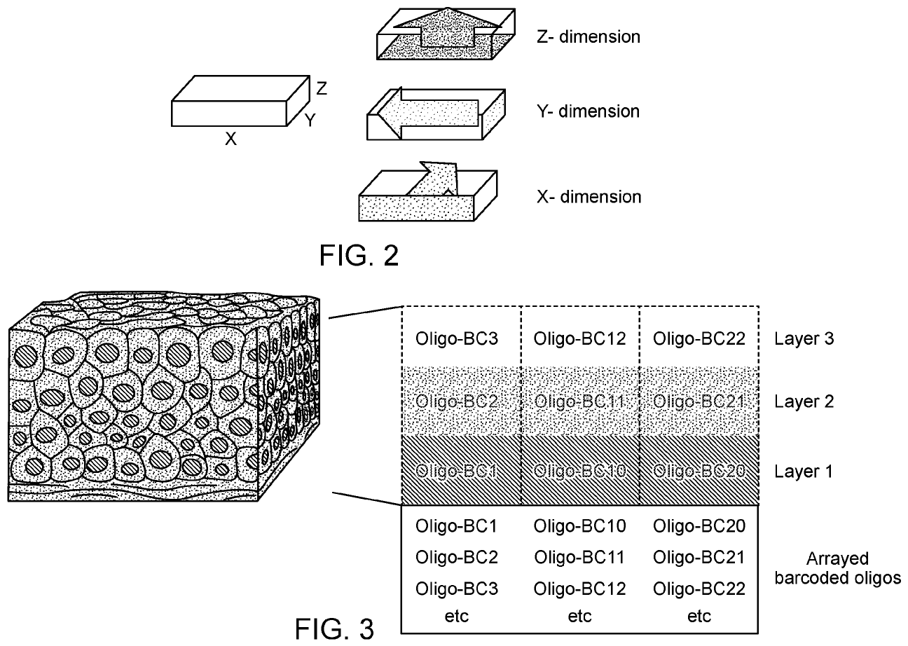

The method of the present disclosure may involve applying a plurality of barcodes to a tissue, where various subsets of the barcodes label different regions of the tissue. Analogous to gel electrophoresis or gel mobility shift assays, different barcodes migrate at different velocities, thereby marking different locations in a specific direction (x, y, or z). FIG. 1 shows some of the principles of the methods and compositions. FIG. 2 shows some examples, but not limited, of the different dimensions the methods can be applied to. In some examples, the barcodes are on a solid support in an array format, with each arrayed element having a unique set of barcodes, e.g. nucleic acids. See FIG. 3 for an example.

The methods and compositions include labeling of analytes with barcodes across a tissue, comprising an analyte capture region (polyA, sequence of the analyte itself, primer site, oligo-tag from antibody, common sequence, common sequence from transposon, etc.), a capture region of the barcodes, wherein the capture region of the analyte and capture region of the barcode are complementary, and a fixation method to maintain location of the analytes in the original sample, and diffusion or active transport of the barcode across the tissue, necessitating the ability to differentially transport barcoding substrates relative to those native in the tissue. This fixation method can be any method in which the analytes are fixed into the original location disallowing diffusion during sample processing. The fixation method can be reversible or non-reversible in nature. The barcode can be attached to the analyte of interest through chemical or enzymatic coupling, ligation, ligation and extension, polymerization, reverse transcription, or any known molecular biology method that links a nucleic acid to an analyte or converts it into a library that can be sequenced. In some examples, a common sequence of the barcode hybridizes to the analyte or derivative of the analyte and binds. In some embodiments, the barcode may contain a fluorescent tag that can be read by microscopy. In some embodiments, the local concentration of the barcode to label the analyte can be 1 nM, 10 nM, 100 nM, 1 uM or higher to efficiently convert the analyte into a barcoded analyte or derivative.

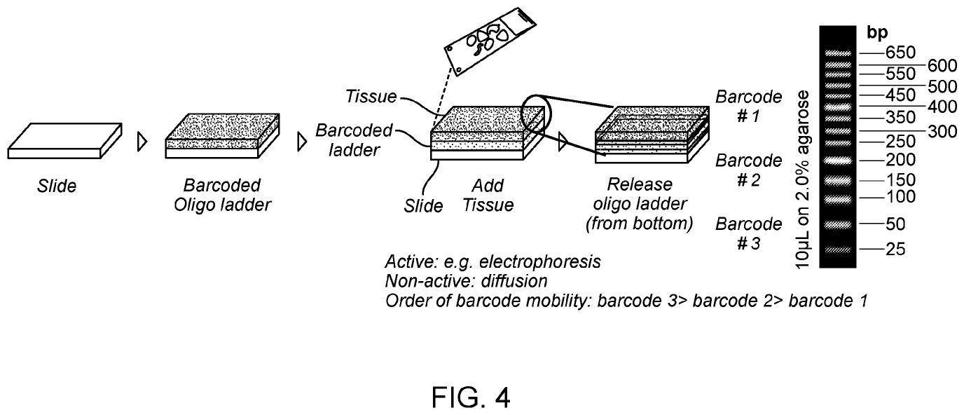

Different barcodes can contain different fluorescent tags that can be distinguished from one another. In some examples, the fluorescent tag provides a reference marker where the cellular sample was barcoded. The present disclosure also provides a method for spatial detection and analysis of nucleic acids in a tissue sample that includes (a) providing a pool of barcodes, comprising a capture site to bind to analytes of interest, wherein a first barcode comprises a first primer binding region and a spatial address region, and wherein a second capture probe comprises a second primer binding region for an analyte that will then encode additional spatial information to the analyte of interest or additional information about the origin or species of that analyte. An example workflow is described in FIG. 4.

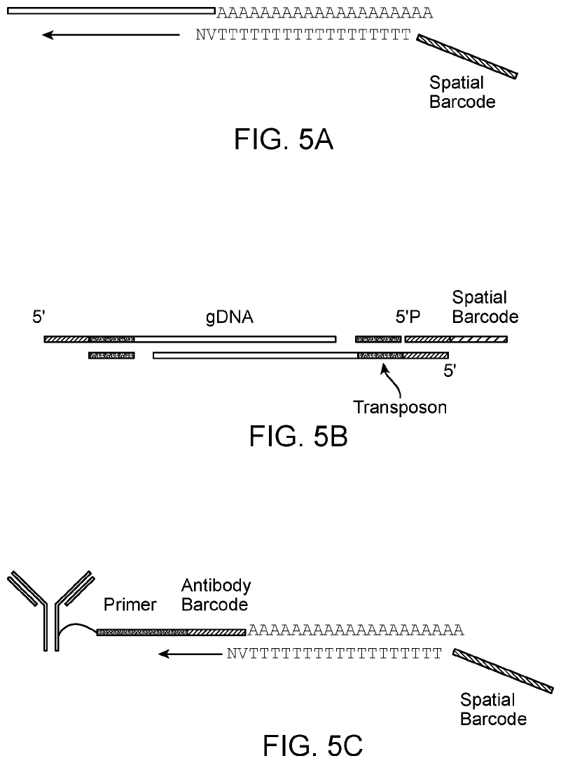

One aspect is that the first and second barcode can originate at the same original spatial location (e.g. z-plane), but through diffusion or active transport label two different unique spatial locations (x,y,z). Each of these independent labeling events can represent unique dimensions spatially and the approach can be extended to a third labeling site that will represent encoding of additional information as to the organization of analytes relative to one another. This is particularly useful in scenarios where the sample does not accommodate efficient labeling by other methods disclosed in this filing, where light patterning, chemical patterning, or other forms of externally interrogative patterning of barcoded substrates to encode information onto an analyte of interest with the sample are limited. The present disclosure also provides a method for spatial detection and analysis of nucleic acids, proteins, DNA, RNA or any combination in a tissue sample (see FIG. 5).

A capture site is defined as any method for encoding or utilizing natural information onto an analyte of interest or of the analyte itself and can be introduced through modification of the analyte through adapter, common or anchor sequence attachment, or primer attachment, transposition, ligation, extension etc. The present disclosure also provides a method for spatial detection and analysis of nucleic acids in a tissue sample that includes one or more barcoded oligonucleotides of different length, composition, molecular modifications, molecular weight, or shape, thereby providing differential spatial orientation to one another. This could be read out, but is not limited to, mobility shifts in the substrate. This disclosure also provides methods around generation of appropriate substrates through chemical, biological, or physical modification of the original molecules to be transported throughout the sample allowing for differential mobility and encoding of spatial information onto the analytes of interest. In some embodiments, this will take the form of random fragmentation of the original capture analytes to be transported to form a pool of capture sequences that will migrate differentially to one another, in others this will take the form of an array of additive methods to the original substrate to enable the differential transport throughout the sample or differential labeling of analytes within the sample. In other embodiments, this will take the form of chemical modification or physical modification of the substrate to act as the barcode that can then be read out post sample processing.

Any method that allows for generation of a pool of capture sequences with a common origin that generates through passive or active transport differential spatial positional information encoded onto analytes of interest could be used. The present disclosure also provides an array of immobilized barcoded nucleic acids on a surface. In the preferred embodiment, the barcoded nucleic acids do not migrate prior to tissue assembly on the surface. In some embodiments, the barcoded nucleic acids are temporarily immobilized on the surface through affinity capture, chemical immobilization, or polymer embedment. The barcoded nucleic acids can be released from the surface through an event including light, chemical or enzymatic cleavage, or any known method to a worker skilled in the art. In some embodiments, the barcoded oligonucleotide can be evenly distributed across the surface that interfaces with the tissue or only interfaces with a section of the tissue. In some embodiments, different barcoded nucleic acids can label different sections of the tissue. In some embodiments the nucleic acids on the surface can be modified in situ to accommodate differential spatial labeling in a region of interest. In some embodiments the immobilized material on the common surface are not nucleic acids that can also be modified in situ for later differential labeling by nucleic acids or other substrates and would migrate differentially either through passive or active transport. In some embodiments, the barcoded nucleic acids can be further modified and barcoded through methods and compositions described in patent application PCT/US2022/019382 entitled ‘Method for adding spatially-addressable barcodes to nucleic acids of a cellular sample in situ’, and is included in its entirety with this application. For example, barcoded nucleic acids distributed over a cellular sample can be further barcoded using reversible terminator synthesis, e.g., by a method that involves, e.g., (a) obtaining a cellular sample comprising nucleic acid molecules that are protected by a reversible terminator; (b) deprotecting the nucleic acid molecules in a set of areas of the sample by selectively applying an external stimulus to the set of areas to produce deprotected nucleic acid molecules in the areas; (c) applying a reversible terminator nucleotide to the cellular sample, resulting in addition of a reversible terminator onto the deprotected nucleic acid molecules; (d) optionally removing any unreacted reversible terminator nucleotide after step (c); and (e) repeating steps (b)-(d) one or more times, to produce spatially addressed barcodes that are attached to nucleic acid molecules that are in or on the cellular sample.

In some embodiments, the sample can be embedded in a polymer. Many different polymers can be used including natural polymers (cellulose, shellac etc.), acrylate, polyacrylamide, agarose, alginate, shape memory polymers e.g., Huang et al. (J. Biomed. Sci. 2019 26:73), OCT, M-1 or other embedding matrix known to a skilled person in the art. In some configurations only the inside of the cells are polymerized (see Arnaud Chemical and Engineering News 2019 97:16). In some embodiments, the polymerization is reversible. The tissue is optionally dissociated after embedding and single cell analytes are immobilized spatially at the location of origin. Additional related methods that could be used are described in Hughes et al. (Nature Methods 2014 11:749-55). In some embodiments the tissue after embedding is cleared, a clearing procedure is used, or some cellular material is removed. Tissue clearing refers to a collection of techniques that render biological samples transparent. Clearing methods typically rely on immersing the tissue in different solutions (like an organic solvent or an aqueous solution with a high refractive index) or possibly embedding the tissue in a hydrogel before extracting tissue lipids with detergents. These techniques enable the deep imaging of large volumes of tissues using light microscopy approaches that are usually limited by the scattering of light by the tissue. As such, clearing methods can improve the accuracy of light deprotection methods as described in this application. Additionally, clearing or removal of cellular material can help with accessibility to the analytes of interest. Many methods and approaches are known to a skilled person in the art including Chung et al. (Nature 2013 497: 332-37), Richardson et al. (Nature Reviews Methods Primers 2021 1:84), Ueda et al (Nature Reviews Neuroscience 2020 21:61-79), Weiss et al. Nature Protocols 2021 16:2732-2748), Ariel (Int J Biochem. Cell Biol. 2017 84:35-39), Chung et al. (Nature 2013 497:332-337) and references cited in the papers. Tissue clearing can be accelerated using electrophoretic approaches like ACT-PRESTO (Lee et al. Scientific Reports 2016 6:18631. Tissue fixation protocols that have been used for expansion microscopy can also be used. These methods use standard cross-linked acrylamide: bis-acrylamide rather than the copolymer with sodium acrylate (which leads to expandability). Sec, e.g., Asano et al (Current Protocols in Cell Biology 2018 80: c56). Some methods may be adapted from single cell western techniques (see, e.g., Hughes ct al Nature Methods 2-14 11:749-55) and further tissue clearance approaches are described in Chung et al. (Nature 2013 497:332-37).

The analytes can be immobilized chemically, non-chemically, through entrapment, covalently or non-covalently to the polymer matrix. After immobilization of the analytes to the polymer matrix, the tissue can be dissociated. One advantage of such approach is that barcode migration in the otherwise heterogeneous nature of the cellular sample can be eliminated, enabling uniform and efficient barcode labeling to each section of the tissue. In some embodiments, the polymer embedded cellular sample may be subjected to expansion, stretching, or enlargement before barcode labeling. In some embodiments, the expansion is only used during barcode addition and no spatial information is directly recorded during time of expansion. Methods and compositions of expansion in the context of spatial single cell analysis have been described in the literature (Alon et al., Science 371, 481, and references cited herein). In some embodiments, the barcode labeling can be performed in any direction, including the x, y, z direction. In some examples, labeling can be performed in one direction, followed by additional labeling in another direction. This process can be repeated many times as desired. After labeling, the label or barcode can be attached to the analyte. The barcode can be different between the different directions, and barcodes can be added to the same target or different targets, for example, target is labeled with barcode from the X direction, then labeled again with a barcode from the y-direction etc.

In some embodiments, the sample, analytes or derivates of analytes (e.g. cDNA, antibody-oligo conjugate bound to a protein etc) are amplified prior to barcoding. For example, nucleic acids can be circularized and rolling circle amplified prior to barcoding thereby making multiple copies to the analyte or derivative of the analyte. In some examples, the analyte or derivative is amplified using a circular template. Method of amplification can include, but not limited, exponential amplification (e.g. PCR), linear amplification (e.g. T7 polymerization), and can include modifications to attach the analytes or derivatives to a matrix in order to maintain its original or close to its original location. In some examples, the pre-processing of the analytes or derivatives prior to barcoding involves linking the analytes or derivatives to a matrix to maintain its original, or close to original, spatial location.

After barcode labeling, the barcoded cells and analytes can be retrieved by extracting the cellular sample or active transport of the barcoded libraries out of the cellular sample. In some embodiments, the barcoded libraries are amplified prior to extraction. Amplification can include PCR, isothermal-, linear amplification-, or rolling circle amplification or any type of amplification of nucleic acid signal.

In some embodiments, the cellular sample is contained in a flow cell or microfluidic device to aid reagent addition and wash steps. In some embodiments, the flow cell is sub-divided into multiple sections enabling the delivery of 2 or more different reagents and solutions, barcodes, terminators, etc per barcode cycle. Example figure https://medical-technology.nridigital.com/medical_technology_nov19/imt_company_insight

After the nucleic acids in or on the sample have been barcoded using the method described above, the barcoded nucleic acids collected, sequenced en masse, and the sequences may be mapped to a position in the sample using the appended barcodes. The barcode serves as an address for the sequence. In some embodiments, the sequences from a particular cell in the sample can be resolved from sequences from other cells in the sample. This concept is illustrated in FIG. 4. In these embodiments, the method may further comprise sequencing the barcodes produced and at least part of the nucleic acid molecules to which they are attached, or an amplification product thereof, and mapping the sequenced nucleic acid molecules to a site in or on the cellular sample using the barcode to which it is attached. The barcoded nucleic acids can have a primer binding site, thereby facilitating amplification of the barcoded nucleic acids. Alternatively, the barcoded nucleic acids may have an affinity tag, thereby facilitating their enrichment. In some embodiments, a subset of the barcoded nucleic acids may be enriched and sequenced, e.g., by enriching for barcoded nucleic acids that have a particular barcode, or nucleic acid sequence. In some examples, the distribution of the barcodes over the sample is known, e.g. barcode 1 is distributed over the sample to a known x1, y1,zl location and barcode 2, is distributed to a known x2, y2,22 location. The sequenced barcoded libraries can be mapped based on the barcode and the known distribution location to the sample. In some of the examples, the location can be a range, e.g. between two coordinates. In some examples, the location of the barcode is recorded through imaging (e.g. fluorescent barcode, fluorescent complement, fluorescent affinity tag to barcode, etc). The barcoded nucleic acids are sequenced, the barcoded nucleic acids are identified (DNA, RNA, mutation, variant, number of transcripts etc) and assigned to the spatial location based on the recording (e.g. fluorescent tag).

The barcoded nucleic acids may be sequenced by any suitable system including Illumina's reversible terminator method, Roche's pyrosequencing method (454), Life Technologies' sequencing by ligation (the SOLID platform), Ultima Genomics (e.g. UG100™), singular genomics (e.g. G4 system), element biosciences (e.g. Aviti™ system), Life Technologies' Ion Torrent platform or Pacific Biosciences' fluorescent base-cleavage method and any other platforms e.g. Oxford Nanopore. Examples of such methods are described in the following references: Margulies et al (Nature 2005 437:376-80); Ronaghi et al (Analytical Biochemistry 1996 242:84-9); Shendure (Science 2005 309:1728); Imelfort et al (Brief Bioinform. 2009 10:609-18); Fox et al (Methods Mol Biol. 2009; 553:79-108); Appleby et al (Methods Mol Biol. 2009; 513:19-39) English (PLOS One. 2012 7: e47768) and Morozova (Genomics. 2008 92:255-64), which are incorporated by reference for the general descriptions of the methods and the particular steps of the methods, including all starting products, reagents, and final products for each of the steps.

The sequencing step may be done using any convenient next generation sequencing method and may result in at least 10,000, at least 100,000, at least 500,000, at least 1M at least 10M at least 100M, at least 1B or at least 10B sequence reads per reaction. In some cases, the reads may be paired-end reads.

Kits for performing the method are also provided. In some embodiments, a kit may comprise a support for a sample that contains the barcoded nucleic acids and a device to distribute the barcoded nucleic acids over the sample. A kit may further contain any of the reagents used in the method or any combination there. A kit may further contain instructions for using the reagents in the present method.

### EXAMPLES

To further illustrate some embodiments of the present invention, the following specific examples are given with the understanding that they are being offered to illustrate examples of the present invention and should not be construed in any way as limiting its scope.

### Example 1

### Embedding Cellular Tissue in Polyacrylamide Gel Matrix and Tissue Clearing.

### Embedding Cellular Tissue

Tissue preparation. Tissues can be fixed using a variety of protocols. For instance, the sample may be fixed by immersion or perfusion using 4% (w/v) paraformaldehyde (PFA) in PBS. To preserve more ultrastructure, PFA and a small percentage of glutaraldehyde (e.g., 4% (w/v) PFA plus 0.1% (w/v) glutaraldehyde in PBS) can be used. After fixation, tissues can be cut to desired length and thickness using a vibratome or cryostat after tissue cryoprotection. The tissue is then taken out and frozen using dry ice and 2-methylbutane. After embedding in polyacrylamide, the sample is ready to be sliced using a cryotome. Prepare polymerization stock solution, monomer solution, and polymerization initiators. Replace the sample buffer with polymerization solution. Leave the tissues in polymerization solution for>6 hr at room temperature with no shaking. Construct the gelation chamber. An example of gelation chamber, assembly, and process is described by Asano et al (Current Protocols in Cell Biology 2018 80: e56). After incubating the tissues in the polymerization solution, the method involves washing the tissues twice, each time for 15 min, with PBS. The gelling solution is then added. Polymerization initiation solution should be added to the solution last. The gelation chamber is then placed in an incubator at 37° C. for 2 hr for polymerization.

### Tissue Clearing

A tissue clearing solution is prepared and the embedded tissue is incubated in the solution for 30 min. An example of a tissue clearing solution is described in Asano et al. Additional examples are described Kurt et al (Nature Protocols 2021 16:2732-2748).

### Example 2

Label Analytes (RNA or Proteins) within Embedded Tissue with Oligonucleotide Anchor Sequence

In this example, proteins are labeled (see FIG. 5C). Proteins are labeled through staining with antibody-conjugated oligonucleotides. The oligonucleotide attached to the antibody serves as a receiving site for the barcoded oligonucleotides distributed using electrophoresis. After embedding the tissue, the tissue is sliced and the slices are used in the next step of the protocol. The tissue can now be stained e.g., immunostaining/immunohistochemistry after fixation, follow a protocol of choice. For example, the fixed tissue can be permeabilized by applying 0.1% (w/v) Triton X-100 in PBS at room temperature for 15 min, then in blocking buffer at room temperature for 6 hr. After this step, the tissue is incubated with oligonucleotide-conjugated primary antibodies in the blocking buffer. The incubation can be performed on a shaker at low speed overnight at room temperature or at 4° C. Next, the antibody solution is removed, and the sample is washed four times, each time for 30 min with blocking buffer, to remove unbound primary antibodies. Proceed to the gelation steps immediately.

After stopping the electrophoresis, barcoded oligonucleotides are captured onto the anchor sequences of the antibody-oligonucleotide conjugates (or alternative to a tag on the oligonucleotide depending on design of the oligonucleotide). After capture, the embedded tissue is washed with a polymerase buffer. After washing, the polymerase master mix is added and a copy of the barcode is made. See FIG. 5C.

### Example 3

### Design of the Electrophoretic System.

Z-directional electrode system. The z-directional electrode separation system consists of planar electrodes integrated into a custom laser-fabricated acrylic alignment setup and brought into contact with two 32 mm diameter, 3 mm-thick neodymium rare-earth magnets on the backside of each electrode (each magnet specified to provide 19 lbs of pull force). The planar electrodes are commercial platinum-coated electrotransfer anodes (Bio-Rad Criterion anode plates). Electric fields are provided by a power supply (Bio-Rad PowerPac® Basic) connected to the electrodes. Temperature control on the electrode maintains gel temperature at ˜4° C. to help to mitigate deleterious effects of heating during separations. An example of a system that could be used for this step is described in Grist et al (Nature Communications 2020 11:6237)

### Example 4

Electrophoretic Distribution of Spatial Barcodes in Z-Axis within Embedded Tissue

Barcoded oligonucleotide array preparation. Barcoded oligonucleotides containing an amino group, a c6 carbon or a PEG spacer, and a cleavable group (e.g., uracil or photocleavable (PC) linker) are spotted onto aminosilane-coated slides using either a GMS 417 spotter (Affymetrix) or a SDDC Microarray spotter (Engineering Systems Inc.). A volume of 50 pL of oligonucleotide solution (30-100 μM) is spotted on the substrate surface. Each spot contains multiple oligonucleotides of different sequence length or incorporates a different “drag tag” (as described in U.S. Pat. No. 9,221,863B2 included by reference) so they can be electrophoretically separated and distributed across the Z-axis and incorporated into the embedded sample. Attachment is achieved by incubating the coated slides with the spotted oligos for 4 h at 60° C. and 10 min at 120° C.

The polymer-embedded tissue is placed on top of the barcoded oligonucleotide slide array and placed into the electrophoretic system. To run the barcode oligonucleotide separations, the anode and embedded tissue gel are stacked. The surface of the gel facing the barcoded array can be optionally dried before stacking to minimize diffusion of barcoded oligonucleotides (if excess or non-bound oligo nucleotide material is present. Barcoded oligonucleotides are released by light through cleaving of the PC linker (UV, 300-400 nm). Constant current between the anode and cathode for varying electrophoresis times is run to barcode the analytes inside the tissue embedded gel. Electrophoresis distributes the barcoded oligonucleotide population based on size. Electrophoresis is stopped and barcoded oligonucleotides are spatially captured through hybridization capture with mRNA or Ab-oligonucleotide-stained proteins, as illustrated in FIG. 5.

### Example 5

Attachment of Spatial Barcodes to mRNA Analytes.

In this example, RNA is labeled (see FIG. 5A). This method involves reverse transcribing RNA in embedded tissue or cells. In this example, the embedded oligonucleotide barcoded tissue is washed with 0.1×SSC buffer (Thermo, AM9770) supplemented with 0.05 U/μl RNase inhibitor (NEB, M0314L). The RNA and barcode oligonucleotide hybrid is reverse transcribed overnight at 42°° C. using SuperScript II (Invitrogen, 18064-014, 10 U/μL reverse transcriptase, 1 mM dNTPs, 1 M betaine solution PCR reagent, 7.5 mM MgCl2, 5 mM DTT, 2 U/μLRNase inhibitor, 2.5 μM TSO and 1× First-Strand buffer. After reverse transcription, embedded tissue is washed twice with 0.1×SSC buffer and incubated with denaturing cDNA removal solution. The solution is neutralized and prepared for amplification. The resulting cDNAs is amplified with KAPA HiFi Hotstart ReadyMix (Roche, KK2602) with 0.8 μM cDNA-PCR primer. PCR reactions is conducted as: first incubation at 95° C. for 5 minutes, 15 cycles at 98° C. for 20 seconds, 58° C. for 20 seconds, 72° C. for 3 minutes and a final incubation at 72° C. for 5 minutes.

### Example 6

### Attachment of Spatial Barcodes to Protein Analytes Via Pre-Attached Anchor Sequences.

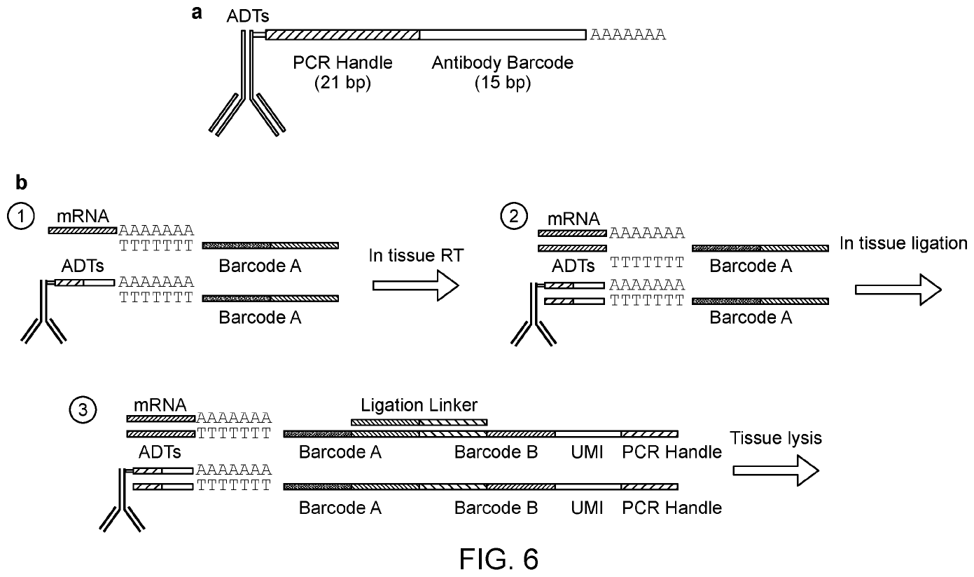

In this example, proteins are indirectly labeled via binding of DNA anchor-tagged antibody binding within fixed-permeabilized tissue sections (as describe in Fan et al Res Sq. 2022 rs.3.rs-1499315). Sections are fixed with 4% formaldehyde for 20 minutes and washed three times with 1× PBS with 0.05U/μL RNase Inhibitor (Enzymatics, 40 U/μL). The tissue is then permeabilized with 0.5% Triton X-100 in 1× PBS for another 20 minutes before washing three times with 1× PBS. The sections are quickly dipped in RNase free water and dried with air. The tissue is then covered with 1× blocking buffer with 0.05U/μL RNase Inhibitor (Enzymatics, 40 U/μL) and incubated at 4° C. for 10 minutes. After washing three times with 1× PBS buffer, DNA-labeled antibody cocktails (diluted 20 times from original stock) from Biolegend (San Diego, CA) are added onto the tissue and incubated for 30 minutes at 4° C. . . . The antibody cocktail is removed by washing three times with 1×PBS. After completion of antibody staining, the spatial barcodes are distributed in the tissue section using the technique of array cleavage of the spatial barcodes underlying the tissues sections and subsequent electrophoresis as described in Example 4. Local proximity of the spatially distributed barcodes enables hybridization and subsequent primer extension on the adapter sequence from the DNA-tagged antibodies, effectively writing the spatial barcode onto the antibody DNA tags. The use of a polyA adapter sequence on the DNA tagged antibodies enables a multi-omics RNA and Protein spatial sequencing co-assay (Fan et al, 2022). See FIG. 6. In this example, the antibody that is conjugated to the antibody has three functional regions: a PCR handle, an antibody barcode and poly-A region. See panel (a). Oligonucleotides and mRNA with Poly-A region at the 3′ end can be reverse transcribed into cDNA using barcode A as the RT primer. Barcode A contains three functional regions, the poly-T region, spatial barcode region and the ligation region.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for spatially labeling analytes in or on a cellular sample in situ, comprising:
(a) obtaining a cellular sample comprising nucleic acid analyte molecules;
(b) contacting the sample with a population of barcoded nucleic acids;
(c) applying a stimulus to distribute the barcoded nucleic acids through the sample, resulting in differentially labeling of the cellular sample;
(d) reacting the barcoded nucleic acids with the nucleic acid analyte molecules, in situ, to produce barcoded nucleic acid products;
(e) extracting the barcoded nucleic acid products from the sample; and
(f) analyzing the extracted barcoded nucleic acid products.

2. The method of claim 1, wherein step (f) comprises sequencing the barcodes and at least part of the nucleic acid molecules to which they are attached, or an amplification product thereof.

3. The method of any prior claim, wherein in step (c) the barcoded nucleic acids are distributed through the sample by diffusion or active transportation.

4. The method of claim 2 or 3, further comprising mapping the sequenced nucleic acid molecules to a site in or on the cellular sample using the barcode to which it is attached.

5. The method of any prior claim, wherein the cellular sample is a tissue or tissue section.

6. The method of claim 1, wherein in (c) barcoded nucleic acids are distributed through the sample in the x, y and/or z plane.

7. The method of any prior claim, wherein the cellular sample of (a) is obtained by hybridizing, ligating, extending, or binding a barcoded oligonucleotide to a sample.

8. The method of any prior claim, wherein the barcoded nucleic acids have different diffusion constants or electrophoretic mobility, thereby uniquely labeling regions of the sample.

9. The method of any prior claim, wherein the population of barcoded nucleic acids comprises barcoded nucleic acids of different lengths and the barcodes indicate the length of the nucleic acid in which it is present and, in step (c), shorter barcoded nucleic acids migrate through the sample further than longer barcoded nucleic acids.

10. The method of any prior claim, wherein barcoded nucleic acids become distributed through the sample according to their lengths or the presence of a drag tag.

11. The method of any prior claim, wherein the cellular sample of (a) is made by:
(i) embedding the cellular sample in a matrix
(ii) fixing the analytes to the matrix
(iii) optionally clearing of cellular materials wherein the matrix provides a medium for transporting barcoded nucleic acids.

12. The method of any prior claim, wherein the barcoded nucleic acids are bound to the analyte or derivative by hybridization.

13. The method of any prior claim, wherein in step (b) the barcoded nucleic acids are initially present on a surface that is in contact with the sample.

14. The method of any prior claim, wherein in step (d) the addition of barcoded nucleic acids is templated.

15. The method of any prior claim, wherein in step (d) the addition of barcoded nucleic acids is non-templated.

16. The method of any prior claim, wherein the barcoded nucleic acids are attached through ligation, extension and/or ligation, PCR, thereby incorporating the barcode into the library.

17. The method of any prior claim, wherein step (d) comprises a ligation, primer extension, gap-fill/ligation, hybridization, and/or chemical ligation.

18. The method of claim 16 or 17, wherein the addition of the barcoded nucleic acid to the analyte or derivative is done enzymatically or chemically.

19. The method of claim 18, wherein the addition is non-templated,

20. The method of claim 18, wherein the addition is templated.

21. The method of any prior claim, wherein in step (c) the barcoded nucleic acid molecules become immobilized in the sample.

22. The method of any prior claim, wherein method comprises binding the plurality of barcoded oligonucleotides to the sample after they are distribution across the sample.

23. The method of any prior claim, wherein population of barcoded nucleic acids comprises at least two barcoded nucleic acids are distributed to different regions in the sample.

24. The method of any prior claim, wherein the number of barcoded nucleic acids comprises at least 10, at least 100, at least 1000, at least 10,000, at least 100,000, at least 1M nucleic acids.

25. The method of any prior claim, wherein the external stimulus applied is electrophoresis, magnetism, temperature, light, pH, or any combination thereof.

26. The method of any prior claim, wherein the external stimulus is selectively applied in one direction to the sample.

27. The method of any prior claim, wherein steps (b)-(c) are repeated at least once.

28. The method of any prior claim, wherein the barcoded nucleic acids of step (b) have different mobilities in the sample.

29. The method of any prior claim, wherein the barcoded nucleic acids of step (b) from one another based on oligonucleotide length, modification, chemical group or combination thereof.

30. The method of any prior claim, wherein the cellular sample of (a) is made by:
i. hybridizing a tailed reverse transcription primer to RNA in the cellular sample;
ii. binding a barcoded oligonucleotide to the reverse transcription primer in situ to produce barcoded first strand cDNA; and
iii. coupling the barcoded oligonucleotide to the cDNA thereby producing barcoded cDNA molecules.

31. The method of claim 30, wherein the barcode is attached to cDNA by ligation or extension and ligation.

32. The method of claim 30, wherein the barcoded nucleic acid contains a common sequence.

33. The method of any prior claim, wherein prior to step (b) the nucleic acid molecules in the sample are either endogenous or produced as a result of transposition, cDNA synthesis, hybridization of an oligonucleotide to an endogenous RNA or DNA, or binding of antibody-oligonucleotide conjugates to endogenous proteins.

34. A kit comprising:
a) a support for a sample that contains the barcoded nucleic acids
b) a device to distribute the barcoded nucleic acids over the sample.

35. The kit of claim 34, further comprising instructions for use in the method of claim 1.
